(19) United States
(12) Patent Application Publication
(10) Pub. No.: US 2008/0287679 A1
(43) Pub. Date: Nov. 20, 2008

(54) Process for preparing clopidogrel

(76) Inventors: Vinod Kumar Kansal, Faridabad (IN); Kompally Praveen, Gajraula (IN); Dhirenkumar N. Mistry, Gujarat (IL)

Correspondence Address: KENYON & KENYON LLP, ONE BROADWAY, NEW YORK, NY, 10004, US

(21) Appl. No.: 12/148,311

(22) Filed: Apr. 18, 2008

Related U.S. Application Data
(60) Provisional application No. 60/925,231, filed on Apr. 18, 2007.

(51) Int. Cl. C07D 495/04

(52) U.S. Cl. 546/114

(57) ABSTRACT

The present invention encompasses processes for the preparation of optically pure clopidogrel camphorsulfonic acid salt without the need to isolate or recover (±) clopidogrel.

### CROSS-REFERENCE TO RELATED APPLICATIONS

This application claims the benefit of U.S. provisional application Ser. No. 60/925,231, filed Apr. 18, 2007, hereby incorporated by reference.

### FIELD OF THE INVENTION

The present invention relates to a process for the preparation of optically pure clopidogrel camphorsulfonic acid salt, in a high yield, which is useful in the synthesis of clopidogrel for the treatment of peripheral arterial diseases.

### BACKGROUND OF THE INVENTION

Clopidogrel (“CLD”), methyl (+)-(S)-α-(2-chlorophenyl)-6,7-dihydrothieno[3,2-c]pyridine-5(4H)-acetate of formula (I),

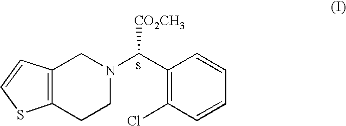

is an inhibitor of adenosine diphosphate (ADP)-induced platelet aggregation which is effective in treating peripheral arterial diseases such as stroke, thrombosis and embolism, as well as coronary arterial diseases such as stroke, thrombosis, embolism, and myocardial infarction. Similar properties are displayed by the less potent racemic mixture (see U.S. Pat. No. 4,847,265).

Clopidogrel is administered as its bisulfate (hydrogen sulfate) salt. Clopidogrel bisulfate has an empirical formula of C₁₆H₁₆ClNO₂S.H₂SO₄. It is currently being marketed as PLAVIX® tablets, which contain about 98 mg clopidogrel bisulfate, which is the equivalent of 75 mg clopidogrel base. PLAVIX® is a white to off-white powder that is practically insoluble in water at neutral pH but highly soluble at acidic pH. It dissolves freely in methanol, somewhat in methylene chloride, and poorly in ethyl ether.

The enantiomer (S) clopidogrel is particularly preferred since it is the pharmaceutically active compound. The enantiomerically enriched compound can be prepared by means of enantioselective synthesis or starting from a racemic mixture of enantiomers in conjunction with a resolution process.

Various methods for preparing clopidogrel are described in U.S. Pat. Nos. 4,847,265, 5,204,469, 6,080,875, 6,495,691, 6,573,381, 6,635,763, 5,132,435, WO2005/104663, and WO 2006/137628.

U.S. Pat. No. 5,132,435 describes a process for the preparation of the (R) and (S) enantiomers of methyl α-(4,5,6,7-tetrahydro-5-thieno[3,2-c]-pyridyl (2-chlorophenyl-) acetate. For example, methyl 1-chloro-(2-chlorophenyl)acetate is coupled with 4,5,6,7-tetrahydrothieno[3,2-c]pyridine in the form of a base or salt to obtain a racemic methyl α-(4,5,6,7-tetrahydro-5-thieno[3,2-c]-pyridyl (2-chlorophenyl-) acetate in the presence of an acid acceptor, e.g. an alkali metal carbonate or bicarbonate, and optionally under phase transfer conditions to obtain the desired racemic clopidogrel base, which has been isolated as clopidogrel hydrochloride salt. The isolated racemic clopidogrel hydrochloride can further be resolved with camphorsulfonic acid in acetone.

The process described in U.S. Pat. No. 5,132,435 requires the isolation of racemic clopidogrel hydrochloride salt (an additional step), resulting in longer reaction cycle time (more than 75 hours) and low yield (33%-39%).

WO 2005/104663 also describes a process for the preparation of racemic clopidogrel. WO 2005/104663 describes a process for resolution of racemic clopidogrel and conversion to hydrogen sulfate salt of clopidogrel via crystalline Forms I and II. The process describes formation of racemic clopidogrel base by coupling 4,5,6,7-tetrahydrothieno(3,2-c)pyridine with methyl-1-halo-(2-chlorophenyl)acetate at room temperature in a solvent, e.g. water and/or dichloroethane in the presence of organic or inorganic bases, e.g. sodium carbonate. Subsequently, resolving the racemic clopidogrel base to provide methyl-(S)-(+)-2-(2-chlorophenyl)-2-(4,5,6,7-tetrahydrothieno[3,2-c]pyridin-5-yl acetate using anhydrous levo-camphor-10-sulphonic acid in a mixture of solvents selected from combination of polar and non-polar/weakly polar solvents like acetone: chloromethane, acetone: toluene, and acetone:cyclohexane. This process also requires isolating the racemic clopidogrel hydrogen sulfate salt prior to resolution, which give rise to longer reaction cycle time and low yield.

U.S. Pat. Nos. 4,529,596, 4,847,265, 5,036,156, 5,189,170 and WO 2006/0137628 refer to various methods of preparing racemic clopidogrel or clopidogrel. These processes also involve the formation of clopidogrel acid salt before its resolution with levorotatory camphorsulphonic acid, which leads to an increase of additional reaction steps, e.g. formation of acid salt and making of free base, thereby increasing (1) the amount of solvents and reagents consumed, (2) reaction cycle time, (3) laborious work ups and separations and (4) effluent load; ultimately results in formation of clopidogrel in poor yield.

U.S. Pat. No. 6,737,411 and US Publication No. 2005/0059696 describe the preparation of clopidogrel hydrogen sulfate.

The present invention provides an improved process for preparing clopidogrel that allows one to obtain an enantiomerically pure or enantiomerically enriched product without the need of laborious procedures and separations.

### SUMMARY OF THE INVENTION

In one embodiment the present invention encompasses a process for preparing (−)-10-camphorsulphonic acid salt of methyl (+)-(S)-α-(2-chlorophenyl)-6,7-dihydrothieno[3,2-c]pyridine-5(4H)-acetate (“CLD-CSA”) of formula II comprising: (a) reacting 4,5,6,7-tetrahydrothieno (3,2-c) pyridine hydrochloride (“formula III”) with o-chlorophenyl-α-bromo methyl acetate (“formula IV”) in the presence of an acid acceptor to produce (±)-methyl α-(2-chlorophenyl)-6,7-dihydrothieno[3,2-c]pyridine-5(4H)-acetate; (b) reacting in-situ (±)-methyl α-(2-chlorophenyl)-6,7-dihydrothieno[3,2-c]pyridine-5(4H)-acetate (“clopidogrel racemate”) with (−)-10-camphorsulphonic acid to afford (−)-10-camphorsulphonic acid salt of methyl (+)-(S)-α-(2-chlorophenyl)-6,7-dihydrothieno[3,2-c]pyridine-5(4H)-acetate of formula II. Preferably, the reaction in step (a) is carried out in a biphasic solvent system optionally under phase transfer conditions. The biphasic (two-phase) system is preferably composed of water and water-immiscible organic solvent mixture.

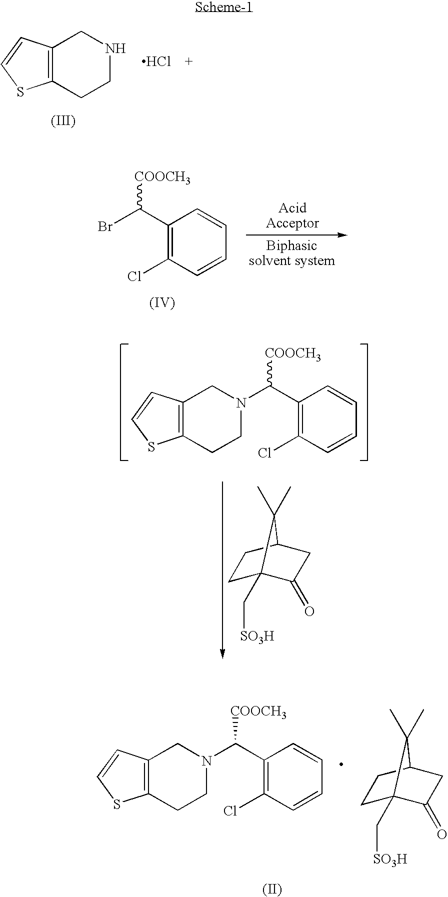

In another embodiment, the invention encompasses a process for preparing (S)-clopidogrel (−)-10-camphorsulphonic acid salt (“CLD-CSA”) comprising: (a) combining (R) clopidogrel or a mixture of (R) and (S) clopidogrel (“formula VI”) with a base to obtain a racemic mixture of (R) and (S) clopidogrel further enriched with (S) clopidogrel (“formula VII”); and (b) reacting the racemic mixture of (R) and (S) clopidogrel further enriched with (s) clopidogrel (“formula VII”) with levorotatory camphorsulphonic acid, to provide (S)-clopidogrel (−)-10-camphorsulphonic acid salt (“formula II”), wherein steps (a) and (b) are carried out without an intermediate step of reacting the racemic mixture of (R) and (S) clopidogrel with sulfuric acid. Preferably, prior to step (a), the process comprises preparing (R) clopidogrel or a mixture of (R) and (S) clopidogrel (“formula VI”) comprising combining mother liquor of (R) clopidogrel (−)-10-camphorsulphonic acid salt or a mixture of (R) and (S) clopidogrel (−)-10-camphorsulphonic acid salt (“formula V”) with a base in an organic solvent to obtain (R) clopidogrel or a mixture of (R) and (S) clopidogrel (“formula VI”).

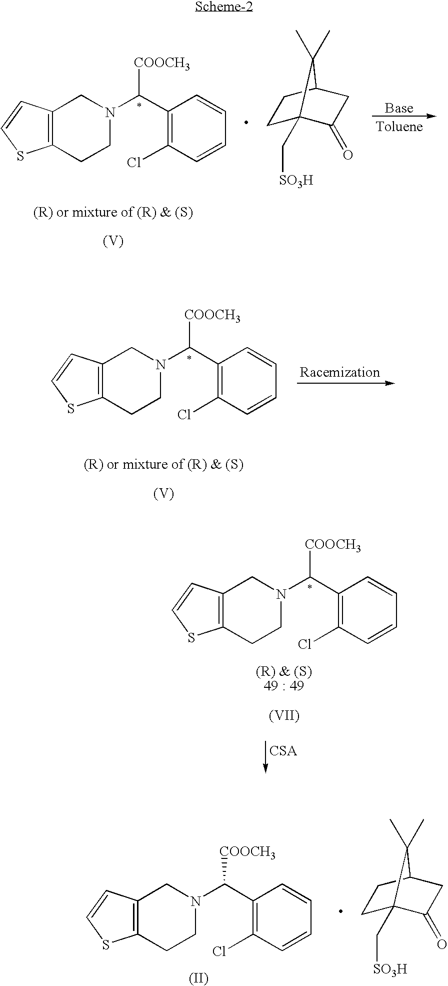

In another embodiment, the invention encompasses a process for the preparation of a pharmaceutically acceptable salt of (S)-clopidogrel from CLD-CSA salt of formula II via conventional techniques. Preferably, the salt is bisulfate.

In another embodiment, the present invention encompasses a process for preparing clopidogrel camphosulfonate comprising: combining 4,5,6,7-tetrahydrothieno-(3,2-c)pyridine hydrochloride, toluene, N,N-dimethyl formamide (“DMF”), o-chlorophenyl-α-bromo methyl acetate to obtain a reaction mixture containing (±) clopidogrel; and converting the (±) clopidogrel to clopidogrel camphosulfonate without the recovery of (±) clopidogrel. Preferably, the process further comprises adding tetrabutylammonium hydrogen sulphate and/or a base to the combination of 4,5,6,7-tetrahydrothieno-(3,2-c)pyridine hydrochloride, toluene, DMF, and o-chlorophenyl-α-bromo methyl acetate.

In another embodiment, the present invention encompasses a process for preparing (S)-clopidogrel bisulfate comprising: (a) reacting 4,5,6,7-tetrahydrothieno (3,2-c) pyridine hydrochloride with o-chlorophenyl-α-bromo methyl acetate in the presence of an acid acceptor to produce (±)-methyl α-(2-chlorophenyl)-6,7-dihydrothieno[3,2-c]pyridine-5(4H)-acetate; (b) reacting in-situ (±)-methyl α-(2-chlorophenyl)-6,7-dihydrothieno[3,2-c]pyridine-5(4H)-acetate with (−)-10-camphorsulphonic acid to provide (−)-10-camphorsulphonic acid salt of methyl (+)-(S)-α-(2-chlorophenyl)-6,7-dihydrothieno[3,2-c]pyridine-5(4H)-acetate, wherein the camphorsulfonate precipitates out of reaction mixture leaving (R)-clopidogrel in the reaction mixture (mother liquor); (c) combining the (R) clopidogrel or a mixture of (R) and (S) clopidogrel remaining in mother liquor with a base to obtain a racemic mixture of (R) and (S) clopidogrel further enriched with (S) clopidogrel; (d) reacting the racemic mixture of (R) and (S) clopidogrel further enriched with (S) clopidogrel with levorotatory camphorsulphonic acid, to provide (S)-clopidogrel (−)-10-camphorsulphonic acid salt, wherein steps (c) and (d) are carried out without an intermediate step of reacting the racemic mixture of (R) and (S) clopidogrel with sulfuric acid; and (e) converting (S)-clopidogrel (−)-10-camphorsulphonic acid salt to (S)-clopidogrel bisulfate.

### DETAIL DESCRIPTION OF THE INVENTION

As used herein, the term “CLD-CSA” refers to (S)-clopidogrel (−)-10-camphorsulphonic acid salt, i.e.: (−)-10-camphorsulphonic acid salt of methyl (+)-(S)-α-(2-chlorophenyl)-6,7-dihydrothieno[3,2-c]pyridine-5(4H)-acetate, of formula II.

As used herein, the term “CSA” refers to camphorsulphonic acid.

As used herein, the term “clopidogrel racemate” refers to (±)-methyl α-(2-chlorophenyl)-6,7-dihydrothieno[3,2-c]pyridine-5(4H)-acetate.

The present invention provides a process for the synthesis of clopidogrel camphorsulphonic acid in one single step, using 4,5,6,7-tetrahydrothieno[3,2-c]pyridine hydrochloride and o-chlorophenyl-α-bromo methyl acetate as starting materials to produce clopidogrel camphorsulphonic acid in high yield with high optical purity without the need to isolate racemic clopidogrel and its pharmaceutically acceptable salts thereof. Preferably, the salt is bisulfate or hydrochloride.

It is observed that the process according to the present invention can provide clopidogrel camphorsulphonic acid in a yield of greater than or equal to 60 wt %, preferably, greater than or equal to about 65 wt %, more preferably, greater than or equal to about 70 wt % (with a chiral purity of about 95% to about 99.9%). Total reaction cycle time can be about 18-22 hours. In addition, applicants have shortened the overall number of steps required for preparing and recovering clopidogrel camphorsulphonic acid; and converting clopidogrel camphorsulphonic acid to clopidogrel bisulfate, preferably to five reaction steps.

The process of the present invention avoids the need to isolate racemic clopidogrel hydrochloride prior to resolution. Thus, by eliminating the isolation step, the present process requires less reaction time, consumes less reagents and solvents, allowing one to obtain an enantiomerically pure or enantiomerically enriched product without the need of laborious procedures and separations. The present process is therefore suitable for industrial scale productions.

In one embodiment, the present invention encompasses a process for preparing (−)-10-camphorsulphonic acid salt of methyl (+)-(S)-α-(2-chlorophenyl)-6,7-dihydrothieno[3,2-c]pyridine-5(4H)-acetate (“CLD-CSA”) comprising: (a) reacting 4,5,6,7-tetrahydrothieno (3,2-c) pyridine hydrochloride (“formula III”) with o-chlorophenyl-α-bromo methyl acetate (“formula IV”) in the presence of an acid acceptor optionally in a biphasic (two-phase) solvent system and under phase transfer conditions to produce (±)-methyl α-(2-chlorophenyl)-6,7-dihydrothieno[3,2-c]pyridine-5(4H)-acetate (“clopidogrel racemate”); (b) reacting in-situ the clopidogrel racemate with (−)-10-camphorsulphonic acid to afford CLD-CSA of formula II (see Scheme 1). Optionally, seeding pure camphorsulfonate after the addition of (−)-10-camphorsulphonic acid in step (b).

The obtained compound of formula II may further be recrystallized in a suitable organic solvent to afford (−)-10-camphorsulphonic acid salt of methyl (+)-(S)-α-(2-chlorophenyl)-6,7-dihydrothieno[3,2-c]pyridine-5(4H)-acetate with preferably about 68% to about 80% yield and high optical purity. The optical purity is at least about 95% to about 99.9%; preferably, at about 98% to about 99.9%.

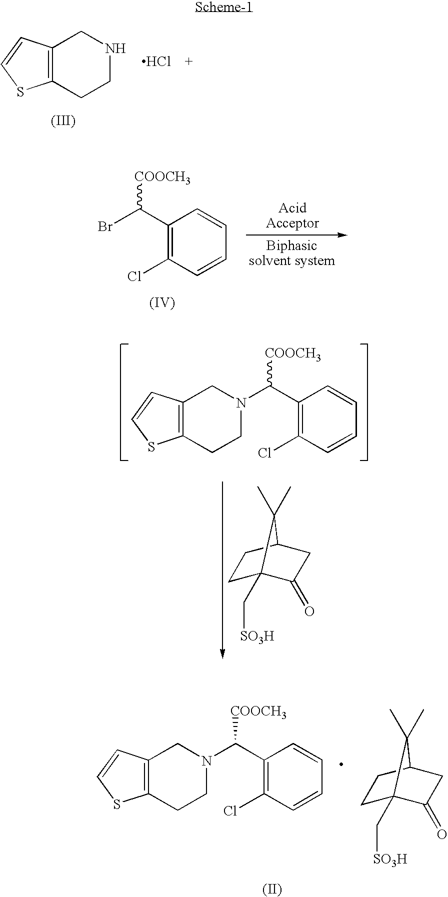

As used herein, unless otherwise defined, the term “phase transfer conditions” means reaction condition which takes place in a two phase (biphasic) solvent system, preferably, with a phase transfer catalyst.

The two phase solvent typically comprises of water and water-immiscible organic solvent. Preferably, the two phase solvent system also contains a suitable co-solvent. Preferably, the co-solvent is selected from the group consisting of dimethyl formamide (“DMF”), dimethyl sulfoxide (“DMSO”), toluene, heptane and dimethylacetamide. Preferably, the co-solvent is present in the range from about 0.2 ml to about 1 ml per gram of compound of formula III.

Preferably, the acid acceptor is a base. More preferably, the acid acceptor is an inorganic base selected the group consisting of alkali metal carbonate and bicarbonate. Most preferably, the acid acceptor is sodium carbonate, potassium carbonate, sodium bicarbonate or potassium bicarbonate. The acid acceptor is employed preferably in amounts ranging from about 1 to about 4 moles per mole of 4,5,6,7-tetrahydrothieno-(3,2-c)pyridine hydrochloride (formula III), more preferably, ranging from about 1 to 3 moles. Most preferably, the acid acceptor is employed in amounts ranging from about 1.5 to about 1.7 moles per mole of 4,5,6,7-tetrahydrothieno-(3,2-c)pyridine hydrochloride (formula III).

Preferably, the water immiscible-organic solvent in biphasic solvent system is employed in the range from about 2 ml to about 10 ml per gram of compound of formula III, more preferably, from about 2 ml to 5 ml. Preferably, water is present in amounts ranging from about 0.5 ml to about 5 ml per gram of compound of formula III, more preferably, from about 0.5 ml to 2 ml.

Typically, the water-immiscible organic solvent is selected from the group consisting of C₆ to C₁₂ aromatic hydrocarbons, halogenated hydrocarbons, C₃ to C₈ ketone, C₃ to C₁₀ alkyl ester, and mixtures thereof.

Halogenated hydrocarbons may include, but are not limited to, cyclic or acyclic, saturated or unsaturated aliphatic or aromatic hydrocarbons. Examples of halogenated hydrocarbons include, but are not limited to, halogenated alkanes (e.g. chloromethane, dichloromethane, chloroethane, dichlorotrifluoroethane, difluoroethane, hexachloroethane, or pentafluoroethane); halogenated alkenes (e.g. tetrachloroethene, dichloroethene, trichloroethene, vinyl chloride, chloro-1,3-butadiene, or chlorotrifluoroethylene); halogenated benzenes (e.g. benzotrichloride, benzyl chloride, bromobenzene, chlorobenzene, chlorotoluene, dichlorobenzene, fluorobenzene, or trichlorobenzene). The preferred halogen is chlorine. The preferred halogenated hydrocarbons are aromatic hydrocarbons or C₁-C₄ alkanes, and more preferably chlorinated aromatic hydrocarbons or C₁-C₄ alkanes. The most preferred halogenated hydrocarbons are chlorobenzene, o- or p-dichlorobenzene, dichloromethane, or o-chlorotoluene.

Typically, the phase transfer catalyst is selected from the group consisting of quaternary ammonium salts, phosphonium salts, crown ethers, and pyridium salt. Examples of suitable quaternary ammonium salts include, but are not limited to, tetraalkylammonium chlorides (e.g. tetramethylammonium chloride, tetraethylammonium chloride, tetrapropylammonium chloride or tetrabutylammonium chloride); tetraalkylammonioum bromides (e.g. tetramethylammonium bromide, tetraethylammonium bromide, tetrapropylammonium bromide or tetrabutylammonium bromide); benzyltrialkylammonium halides (e.g. benzyltrimethylammonium chloride, benzyltrimethylammonium bromide, benzyl-tri-n-butylammonium chloride or benzyl-tri-n-butylammonium bromide); cetyltrialkylammonium halides (e.g. cetyltrimethylammonium chloride, cetyltrimethylammonium bromide, cetyltriethylammonium chloride, or cetyltriethylammonium bromide); tetraalkylammonium hydroxides (e.g. tetramethylammonium hydroxide, tetraethylammonium hydroxide, tetrapropylammonium hydroxide or tetrabutylammonium hydroxide); or benzyltrialkylammonium hydroxides (e.g. benzyltrimethylammonium hydroxide, benayltrimethylammonium hydroxide, benzyltri-n-butylammonium hydroxide or benzyl-tri-n-butylammonium hydroxide). Examples of suitable phosphonium salts include, but are not limited to, phosphonium chloride, phosphonium bromide, trimethylphosphonium chloride, triethylphosphonium bromide, tetramethylphosphonium chloride, tetramethylphosphonium bromide, ethyl triphenyl phosphonium bromide, ethyl triphenyl phosphonium iodide, butyl triphenyl phosphonium bromide, benzyl triphenyl phosphonium chloride, methyl triphenyl phosphonium bromide, methyl triphenyl phosphonium iodide, tetra phenyl phosphonium bromide, methyl triphenyl phosphonium bromide, butyl triphenyl phosphonium chloride, (methoxy methyl) triphenyl phosphonium chloride, or phosphonium iodide. Examples of suitable crown ethers include, but are not limited to, 8-crown-6, or 15-crown-5. Examples of suitable pyridinium salts include, but are not limited to, cetyl pyridinium chloride, cetyl pyridinium bromide, lauryl pyridinium chloride, or dodecyl pyridinium chloride.

Preferably, the phase transfer catalyst is a quaternary ammonium salt. Quaternary ammonium salts are preferred because they are readily available commercially and when used, produce the desired product in high yield. More preferably, the phase transfer catalyst is tetraalkylammonium halides, benzyltrialkylammonium halides, or tetraalkylammonium hydrogen sulfate. Preferably, the phase transfer catalyst is employed in amounts ranging from about 0.01 mole to 0.1 mole per mole of 4,5,6,7-tetrahydrothieno-(3,2-c)pyridine hydrochloride (formula III).

Preferably, o-chlorophenyl-α-bromo methyl acetate (formula IV) is present in amounts ranging from about 0.8 mole to 1.5 moles per mole of 4,5,6,7-tetrahydrothieno (3,2-c) pyridine hydrochloride (formula III).

Preferably, in step (a), the reaction between 4,5,6,7-tetrahydrothieno (3,2-c) pyridine hydrochloride (formula III) and o-chlorophenyl-α-bromo methyl acetate (formula IV) is carried out at a temperature of about 25° C. to about 100° C. More preferably, the reaction temperature is at about 25° C. to about 60° C. As a result, (+)-methyl α-(2-chlorophenyl)-6,7-dihydrothieno[3,2-c]pyridine-5(4H)-acetate is formed in-situ and is further used in the subsequent step without isolation or recovery.

Preferably, in step (b), after adding camphorsulphonic acid, the reaction mixture is maintained, with agitation, for about 2 to about 5 hours at a temperature of about 25° C. to about 45° C. The camphorsulphonic acid reaction mixture is then cooled to a temperature of about 10° C. to about 25° C. and preferably, maintained with agitation for about 2 to about 8 hours.

Preferably, recrystallization of compound of formula II is achieved with the addition of about 3 ml to about 12 ml of organic solvent. Suitable organic solvents include, but are not limited to, halogenated hydrocarbons, C₃ to C₈ ketone, C₃ to C₁₀ alkyl ester, or mixtures thereof. Preferably, the organic solvent is C₃ to C₈ ketone.

As used herein, the term “mother liquor” refers to the filtrate collected after resolution with camphorsulphonic acid and camphorsulfonate precipitates out of the reaction mixture, wherein the filtrate is left with a mixture of (R) and (S) clopidogrel enriched with (R) clopidogrel.

In another embodiment, the present invention encompasses a process for the recovery of (S)-clopidogrel CSA salt from its mother liquor of (R)-clopidogrel CSA salt or a mixture of (R) and (S)-clopidogrel CSA salt without formation of (R)-clopidogrel bisulfate salt or a mixture of (R) and (S) clopidogrel bisulfate salt. Thus, this process improves the product yield and reduces the overall reaction time and the amounts of reagents/solvents consumed.

In a further embodiment, the present invention encompasses a process for preparing (S)-clopidogrel CSA salt (Scheme 2) comprising the steps of: (a) combining (R) clopidogrel or a mixture of (R) and (S) clopidogrel (“formula VI”) with a base to obtain a racemic mixture of (R) and (S) clopidogrel further enriched with (S) clopidogrel (“formula VII”); and (b) reacting the racemic mixture of (R) and (S) clopidogrel further enriched with (s) clopidogrel of formula VII with levorotatory camphorsulphonic acid, to provide CLD-CSA of formula II, wherein steps (a) and (b) are carried out without an intermediate step of reacting compound of formula VII with sulfuric acid. The obtained CLD-CSA can be further purified by recrystallizing CLD-CSA in a suitable organic solvent. Suitable organic solvents include, but not limited to, ketones. Preferably, the organic solvent is acetone.

Preferably, prior to step (a), the process comprises preparing (R) clopidogrel or a mixture of (R) and (S) clopidogrel (“formula VI”) comprising combining mother liquor of (R) clopidogrel (−)-10-camphorsulphonic acid salt or a mixture of (R) and (S) clopidogrel (−)-10-camphorsulphonic acid salt (“formula V”) with a base in an organic solvent to obtain (R) clopidogrel or a mixture of (R) and (S) clopidogrel (“formula VI”).

The present process accomplishes recycling the remaining (R) clopidogrel by racemizing the (R) enantiomer into a mixture of (S) and (R) enantiomers, and separating the two enantiomers as described above. As one of skill in the art would appreciate, the recycling step can be repeated many times to recycle as much of the (R) enantiomer as possible.

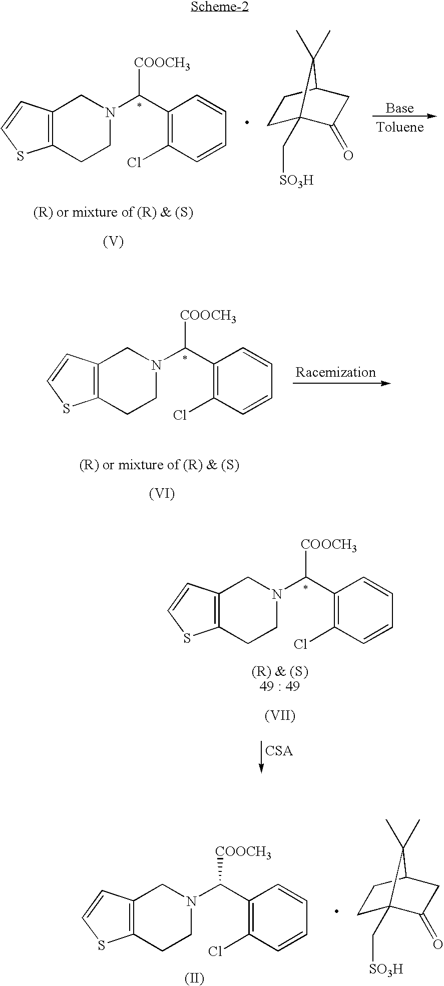

The present process uses bases for the preparation of free base of (R)-clopidogrel or mixture of (R) and (S) clopidogrel of formula VI. One of skill in the art would appreciate that many bases can be used for the preparation of compound VI. Examples of bases include, but are not limited to, for example, an organic amine, an alkali metal alkoxide, an alkali metal hydroxide, an alkaline earth metal hydroxide, an alkali metal hydride, an alkaline earth metal hydride, an alkali or alkaline earth metal carbonate or hydrogencarbonate salt. Specific examples of bases include, but are not limited to, for example, 1,8-bis(N,N-dimethylamino)napthalene, sodium methoxide, sodium ethoxide, sodium phenoxide, sodium hydroxide, potassium hydroxide, calcium hydroxide, magnesium hydroxide, sodium hydride, potassium hydride, calcium hydride, sodium carbonate, potassium carbonate, sodium hydrogencarbonate, potassium hydrogencarbonate, calcium carbonate or basic alumina. Preferably, the base is sodium hydrogen carbonate. Preferably, the base is employed in amounts ranging from about 0.1 to 1.0 mole per 1 liter of mother liquor, more preferably, ranging from about 0.1 to 0.5 moles.

A base can also be used to racemize the (R)-clopidogrel. A preferred inorganic base is sodium/potassium hydroxide, while a preferred organic base is a sodium/potassium C₁ to C₄ alkoxide. A particularly preferred base is sodium t-butoxide or potassium t-butoxide, which is more effective than sodium/potassium methoxides. Bases, particularly alkoxides such as t-butoxide are highly reactive towards moisture, and in order for the t-butoxide added to be effective, the organic phase preferably has low water content. Preferably, the water content of the organic phase is less than or equal to about 0.1%, more preferably, about 0.05%, as determined by the Karl Fischer method. After achieving an acceptable water level, a catalytic amount of potassium t-butoxide is added to the organic phase. Preferably, the base is employed in amounts ranging from about 0.01 to 0.5 moles per 1 mole of compound of formula VI, more preferably, ranging from about 0.01 to 0.1 moles. The molar ratio also generally applies to other bases.

The resolution can be done in a suitable organic solvent. Suitable organic solvent is selected from the group consisting of C₆ to C₁₂ aromatic hydrocarbons, halogenated hydrocarbons, C₃ to C₈ ketone, C₃ to C₁₀ alkyl ester, and mixtures thereof. Preferably, the organic solvent is toluene.

In one embodiment, the CSA is employed in amounts ranging from about 0.1 to 1.0 mole per 1 mole of compound of formula VII, more preferably, ranging from about 0.4 to 0.6 moles.

The (−)-10-camphorsulphonic acid salt of methyl (+)-(S)-α-(2-chlorophenyl)-6,7-dihydrothieno[3,2-c]pyridine-5(4H)-acetate of formula (II) can be converted into clopidogrel or its pharmaceutically acceptable salt by any method known to one of skill in the art, for example, by one of the methods disclosed in U.S. Pat. Nos. 4,847,265 and 5,132,435.

In one example embodiment, the present invention encompasses a process for preparing clopidogrel camphosulfonate comprising: combining 4,5,6,7-tetrahydrothieno-(3,2-c)pyridine hydrochloride, toluene, DMF, o-chlorophenyl-α-bromo methyl acetate to obtain a reaction mixture containing (+) clopidogrel; and converting the reaction mixture containing (±) clopidogrel to clopidogrel camphosulfonate without the recovery of (±) clopidogrel. Preferably, the process further comprises adding tetrabutylammonium hydrogen sulphate and/or a base to the combination of 4,5,6,7-tetrahydrothieno-(3,2-c)pyridine hydrochloride, toluene, DMF, and o-chlorophenyl-α-bromo methyl acetate. Preferably, the solvent ratio between toluene and DMF is 0.8:4.2 by volume.

Prior to conversion, water can be added to form a two-phase system, and the organic layer containing (+) clopidogrel can subsequently be separated. Additional toluene and DMF can be added to the reaction mixture. The organic layer can be separated and seeded with (−)-10-camphorsulphonic acid salt of methyl (+)-(S)-α-(2-chlorophenyl)-6,7-dihydrothieno[3,2-c]pyridine-5(4H)-acetate. Instead of adding toluene and DMF in the first step, other solvent combinations, e.g. dichloromethane and water, ethyl acetate and water, dichloromethane and water, toluene, water and DMF, toluene, water and dimethylsulfoxide, toluene, water and dimethylacetamide can be added.

In another embodiment, the present invention encompasses a process for preparing (S)-clopidogrel bisulfate comprising: (a) reacting 4,5,6,7-tetrahydrothieno (3,2-c) pyridine hydrochloride with o-chlorophenyl-α-bromo methyl acetate in the presence of an acid acceptor to produce (±)-methyl α-(2-chlorophenyl)-6,7-dihydrothieno[3,2-c]pyridine-5(4H)-acetate; (b) reacting in-situ (±)-methyl α-(2-chlorophenyl)-6,7-dihydrothieno[3,2-c]pyridine-5(4H)-acetate with (−)-10-camphorsulphonic acid to provide (−)-10-camphorsulphonic acid salt of methyl (+)-(S)-α-(2-chlorophenyl)-6,7-dihydrothieno[3,2-c]pyridine-5(4H)-acetate, wherein the camphorsulfonate precipitates out of reaction mixture leaving (R)-clopidogrel in the reaction mixture (mother liquor); (c) combining the (R) clopidogrel or a mixture of (R) and (S) clopidogrel remaining in mother liquor with a base to obtain a racemic mixture of (R) and (S) clopidogrel further enriched with (S) clopidogrel; (d) reacting the racemic mixture of (R) and (S) clopidogrel further enriched with (S) clopidogrel with levorotatory camphorsulphonic acid, to provide (S)-clopidogrel (−)-10-camphorsulphonic acid salt, wherein steps (c) and (d) are carried out without an intermediate step of reacting the racemic mixture of (R) and (S) clopidogrel with sulfuric acid; and (e) converting (S)-clopidogrel (−)-10-camphorsulphonic acid salt to (S)-clopidogrel bisulfate.

In another example embodiment, the present invention encompasses a process for preparing (S)-clopidogrel bisulfate comprising: reacting 4,5,6,7-tetrahydrothieno-(3,2-c)pyridine hydrochloride with o-chlorophenyl-α-bromo methyl acetate in the presence of potassium carbonate in a media of toluene, dimethyl formamide and water, to form clopidogrel racemate, after work-up, an organic phase is taken for resolution; to the organic phase, dimethyl formamide and camphorsulfonic acid are added and after maintaining, filter to obtain (−)-10-camphorsulphonic acid salt of methyl (+)-(S)-α-(2-chlorophenyl)-6,7-dihydrothieno[3,2-c]pyridine-5(4H)-acetate, and washed with acetone; then, the filtrate is taken for racemization; isolate (−)-10-camphorsulphonic acid salt of methyl (+)-(S)-α-(2-chlorophenyl)-6,7-dihydrothieno[3,2-c]pyridine-5(4H)-acetate and purify it by refluxing in acetone; pure (−)-10-camphorsulphonic acid salt of methyl (+)-(S)-α-(2-chlorophenyl)-6,7-dihydrothieno[3,2-c]pyridine-5(4H)-acetate taken in ethyl acetate and pH adjusted to 8 to 8.5 with sodium bicarbonate, this was washed with water and phase separation done, discard the lower aqueous layer; distill organic layer under vacuum to provide clopidogrel base, to this, acetone is added to provide a solution and adding sulfuric acid to the solution to produce clopidogrel bisulphate.

Having described the invention with reference to certain preferred embodiments, other embodiments will become apparent to one of skill in the art from consideration of the specification. The invention is further defined by reference to the following non-limiting examples describing in detail the synthesis of (S)-clopidogrel (−)-10-camphorsulphonic acid salt. It will be apparent to those of skill in the art that many modifications, both to materials and methods, may be practiced without departing from the scope of the invention.

### EXAMPLES

### HPLC (High Performance Liquid Chromatography)

Column & Packing XTerra Phenyl 5 micron*4.6*250 mm

Part number 186001147 or equivalent

Column temperature: 25-30° C.

Eluent: dodecyl sulphate sodium salt (5 g) dissolved in 500 ml water, adjust to pH 3.0 with H₃PO₄, 420 ml acetonitrile, and 80 ml methanol

Flow rate: 1.3 ml/minute

Detector: 220 nm

Sample volume: 10 Micro liter

Diluent: Eluent

All pH values were measured using pH meter by Toshniwal, Model CL46

### Example 1

### Preparation of (+) Clopidogrel (−) CSA Salt

100 g (0.57 mole) of 4,5,6,7-tetrahydrothieno-(3,2-c)pyridine hydrochloride was added to a 1000 ml reaction vessel equipped with reflux condenser and over head agitator, to this, 400 ml of toluene was added at a temperature of 20-25° C. and charged with 174 g (1.25 mole) of potassium carbonate, 80 ml of demineralized (“DM”) water, and 100 ml of 80 ml of dimethyl formamide and 10 g of tetrabutylammonium hydrogen sulphate. Stirred the reaction mixture for 30-60 minutes at a temperature of 20-25° C. Subsequently, raised reaction temperature to 30-35° C. and 150 g (0.57 mole) of o-Chlorophenyl-α-bromo methyl acetate was added to the reaction mixture over 1 hour and stirred the reaction mixture for additional 3-4 hours at 30-35° C. The completion of reaction was monitored by HPLC analysis. Then, cooled reaction mixture to 30° C. and 800 ml of DM water was added to form a two phase system and stirred for 0.5 to 2 hrs and two resulting phases were separated. The aqueous phase was extracted with 100 ml toluene and the organic layers were combined. To the organic phase, 300 ml toluene and 50 g of DMF were added and stirred for 30 minutes to obtain a clear solution, to this, 70 g of anhydrous 1-10-Camphorsulphonic acid was added and heated to 40° C. The reaction mixture was seeded with 0.5 g of (−)-10-Camphorsulphonic acid salt of methyl (+)-(S)-α-(2-chlorophenyl)-6,7-dihydrothieno[3,2-c]pyridine-5(4H)-acetate and maintained for 4 hours. Gradually cooled reaction mixture to 15-20° C. and maintained for 4-5 hours, filtered and washed with 2×100 ml of chilled toluene. Then washed with 100 ml of chilled acetone and vacuum dried. Charged the wet compound of (−)-10-Camphorsulphonic acid salt of methyl (+)-(S)-α-(2-chlorophenyl)-6,7-dihydrothieno[3,2-c]pyridine-5(4H)-acetate into a 2000 ml reaction vessel equipped with reflux condenser. To this, added 685 ml of acetone (5.05 volumes wet CLD-CSA) and heated to reflux and maintained for 6 hours. Gradually cooled to 15-20° C., filtered and vacuum dried. Spray washed the wet cake with 300 ml chilled acetone. Dried the wet (−)-10-Camphorsulphonic acid salt of methyl (+)-(S)-α-(2-chlorophenyl)-6,7-dihydrothieno[3,2-c]pyridine-5(4H)-acetate at 40-50° C. under vacuum. Yield of CLD-CSA: 110 g (70% yield) Optical purity: 99.2%.

### Example 2

### Preparation of (+) Clopidogrel (−) CSA Salt

100 g (0.57 mole) of 4,5,6,7-tetrahydrothieno (3,2-c) pyridine hydrochloride was added to a 1000 ml reaction vessel equipped with reflux condenser and over head agitator, to this, 400 ml of dichloromethane was added at 25-30° C. and charged with 174 g (1.25 mole) of potassium carbonate, 80 ml of DM water, 10 g of tetrabutylammonium hydrogen sulfate. Stirred the reaction mixture for 30-60 minutes at a temperature of 25-30° C. Subsequently, raised the reaction temperature to 35-40° C. and 150 g (0.57 mole) of o-chlorophenyl-α-bromo methyl acetate was added to the reaction mixture over 1 hour and maintained for 4 hours at 30-35° C. The completion of the reaction was monitored by HPLC analysis. Cooled reaction mixture to 30° C. and 800 ml of DM water was added to form a two-phase system. Stirred for additional 0.5 to 2 hours and two resulting phases were separated. The aqueous phase was extracted with 100 ml of dichloromethane and combined the dichloromethane layer with the main organic layer. To the organic phase, 800 ml of toluene and 50 g of dimethyl formamide were added and stirred for 30 minutes to obtain a clear solution, to this, charged with 70 g of camphorsulfonic acid and heated to 40° C., then 0.1-1 g of CLD-CSA crystals was added and maintained for 4 hours. Gradually cooled to 15-20° C. and maintained for 4-5 hours, filtered and washed with 2×100 ml of chilled toluene. Then washed with 100 ml of chilled acetone and vacuum dried the compound. Charged wet CLD-CSA salt into a 2000 ml reaction vessel equipped with reflux condenser. To this, added 10-12 volumes of acetone and reaction mixture heated to reflux and maintained for 6 hours. Gradually cooled to 15-20° C., filtered and vacuum dried the compound. Spray washed the wet cake with 500 ml of chilled acetone. Dried the wet CLD-CSA salt at 40-50° C. under vacuum. Yield of CLD-CSA: 107 g (68.15% yield) Optical purity: 99.3%.

### Example 3

### Preparation of (+) Clopidogrel (−) CSA Salt

100 g (0.57 mole) of 4,5,6,7-tetrahydrothieno-(3,2-c)pyridine hydrochloride was added to a 1000 ml reaction vessel equipped with reflux condenser and over head agitator, to this, 400 ml of ethyl acetate was added at 25-30° C. and charged with 174 g (1.25 mole) of potassium carbonate, 80 ml of DM water, and 10 g of tetrabutylammonium hydrogen sulfate. Reaction mixture was then stirred for 30-60 minutes at 25-30° C. Subsequently, raised the reaction temperature to 40-60° C. and 150 g (0.57 mole) of o-Chlorophenyl-α-bromo methyl acetate was added to the reaction mixture over 1 hour and maintained for 4 hours at 35-40° C. Completion of the reaction was ensured by HPLC analysis. Then the reaction mixture was cooled to 30° C. and 800 ml of DM water was added to form a two-phase system and stirred for 30-120 minutes and two resulting phases were separated. To the aqueous phase, 100 ml of ethyl acetate was added and combined the ethyl acetate layer with the main organic layer. Distilled off ethyl acetate under reduced pressure at 40-60° C. To the organic phase, 800 ml of toluene and 50 g of DMF were added and stirred for 30 minutes to obtain a clear solution, to this, 70 g of (−)-10-camphorsulfonic acid was added and heated to 40° C., 0.1 g of CLD-CSA crystals was also added and maintained for 4 hours. Gradually cooled reaction mixture to 15-20° C. and maintained for 4-5 hours, filtered and washed with 2×100 ml of chilled toluene. Then washed with 100 ml of chilled acetone and vacuum dried the compound. Charged wet CLD-CSA salt into a 2000 ml reaction vessel equipped with reflux condenser. Added 10 volumes of acetone and heated to reflux and maintained for 6 hours and gradually cooled to 15-20° C., filtered and vacuum dried. Spray washed the wet cake with 500 ml of chilled acetone. Dried the obtained compound at 40-50° C. under reduced pressure to afford 108 g (68.78% yield) of CLD-CSA salt. Optical purity: 99.2%.

### Example 4

### Preparation of (+) Clopidogrel (−) CSA Salt

20 g (0.11 mole) of 4,5,6,7-tetrahydrothieno-(3,2-c)pyridine hydrochloride was added to a 500 ml reaction vessel equipped with reflux condenser and over head agitator, to this, 100 ml of dichloromethane was added at 25-30° C. and charged with 34.8 g (0.25 mole) of potassium carbonate, 16 ml of DM water and Ig of tetrabutylammonium hydrogen sulphate. Reaction mixture was then stirred for 30-60 minutes at 25-30° C. And raised the reaction temperature to 40-60° C. and 30.0 g (0.11 mole) of o-Chlorophenyl-α-bromo methyl acetate was added to the reaction mixture over 1 hour and maintained for 4 hours at 30-35° C. Completion of the reaction was ensured by HPLC analysis. Reaction mixture was then cooled to 30° C. and 100 ml of DM water was added to form a two-phase system and stirred for 0.5-2 hours and two resulting phases were separated. To the aqueous phase, 20 ml of dichloromethane was added and combined the dichloromethane layer with the main organic layer. Distilled off dichloromethane under reduced pressure at 40-60° C. To the organic phase, 160 ml of toluene, 10 g of dimethyl formamide and 12 g of (−)-10-camphorsulfonic acid were added. 0.1 g of CLD-CSA crystals was also added and maintained for 4 hours. Gradually cooled to 15-20° C. and maintained for 4-5 hours, filtered and washed with 2×20 ml of chilled toluene. Then washed with 20 ml of chilled acetone and vacuum dried the compound. Charged wet CLD-CSA salt into a 500 ml reaction vessel equipped with reflux condenser. To this, added 10 volumes of acetone and heated to reflux and maintained for 6 hours. Gradually cooled to 15-20° C., filtered and vacuum dried. Spray washed the wet cake with 50 ml of chilled acetone. The obtained compound was dried at 40-50° C. under reduced pressure to afford 20 g (63.69% yield) of CLD-CSA salt. Optical purity: 99.1%.

### Example 5

### Preparation of (+) Clopidogrel (−) CSA Salt

20 g (0.11 mole) of 4,5,6,7-tetrahydrothieno-(3,2-c)pyridine hydrochloride was added to a 500 ml reaction vessel equipped with reflux condenser and over head agitator, to this, 100 ml of dichloromethane was added at 25-30° C. and charged with 34.8 g (0.25 mole) of potassium carbonate, 16 ml of DM water and 1 g of tetrabutylammonium hydrogen sulphate. Reaction mixture was then stirred for 30-60 minutes at 25-30° C. Subsequently, raised the reaction temperature to 30-35° C. and 30.0 g (0.11 mole) of o-Chlorophenyl-α-bromo methyl acetate was added to the reaction mixture over 1 hour and maintained for 4 hours at 30-35° C. Completion of the reaction was ensured by HPLC analysis. Cooled reaction mixture to 30° C. and 100 ml of DM water was added to form a two-phase system and stirred for 0.5-2 hours and two resulting phases were separated. To the aqueous phase, 20 ml of dichloromethane extraction was added and combined the dichloromethane layer with the main organic layer. Distilled off dichloromethane under reduced pressure at 40-60° C. To the organic phase, 160 ml of toluene, 10 g of dimethyl formamide and 12 g of camphorsulphonic acid were added. 0.1 g of CLD-CSA crystals was also added and maintained for 4 hours. Gradually cooled to 15-20° C. and maintained for 4-5 hours, filtered and washed with 2×20 ml of chilled toluene. Then washed with 100 ml of chilled acetone and vacuum dried the compound. Charged wet CLD-CSA salt into a 500 ml reaction vessel equipped with reflux condenser. To this, added 10 volumes of acetone and heated to reflux and maintained for 6 hours and gradually cooled to 15-20° C., filtered and vacuum dried. Spray washed the wet cake with 50 ml of chilled acetone. Dried the obtained compound at 40-50° C. under reduced pressure to afford 20 g (63.69% yield) of CLD-CSA salt. Optical purity: 99.2%.

### Example 6

### Preparation of (+) Clopidogrel (−) CSA Salt

20 g (0.11 mole) of 4,5,6,7-tetrahydrothieno-(3,2-c)pyridine hydrochloride was added to a 500 ml reaction vessel equipped with reflux condenser and over head agitator, to this, 100 ml of toluene was added at 25-30° C. and charged with 34.8 g (0.25 mole) of potassium carbonate, 16 ml of DM water, 1 g of tetrabutylammonium bromide and 16.0 ml of DMF. Reaction mixture was then stirred for 30-60 minutes at 25-30° C. Subsequently, raised the reaction temperature to 30-35° C. and 30 g (0.11 mole) of o-Chlorophenyl-α-bromo methyl acetate was added to the reaction mixture over 1 hour and maintained for 4 hours at 30-35° C. Completion of the reaction was ensured by HPLC analysis. Reaction mixture was then cooled to 30° C. and 100 ml of DM water was added to form a two-phase system and stirred for 0.5-2 hours and two resulting phases were separated. To the aqueous phase 20 ml of toluene was added and the organic layers were combined. To the organic phase, 60 ml of toluene, 10 g of DMF and 12 g of camphorsulfonic acid were added. The reaction mixture was seeded with 0.1 g of CLD-CSA crystals and maintained for 4 hours. Gradually cooled to 15-20° C. and maintained for 4-5 hours, filtered and washed with 2×20 ml of chilled toluene. Then washed with 20 ml of chilled acetone and vacuum dried the compound. Charged wet CLD-CSA salt into a 500 ml reaction vessel equipped with reflux condenser. To this, added 10 volumes of acetone and heated to reflux and maintained for 6 hours. Gradually cooled to 15-20° C., filtered and vacuum dried. Spray washed the wet cake with 50 ml of chilled acetone. Dried the obtained compound at 40-50° C. under reduced pressure to afford 20 g (63.69% yield) of CLD-CSA salt. Optical purity: 99.3%.

### Example 7

### Preparation of (+) Clopidogrel (−) CSA Salt

20 g (0.11 mole) of 4,5,6,7-tetrahydrothieno-(3,2-c)pyridine hydrochloride was added to a 500 ml reaction vessel equipped with reflux condenser and over head agitator, to this, 100 ml of toluene was added at 25-30° C. and charged with 34.8 g (0.25 mole) of potassium carbonate, 16 ml of DM water, 1 g of tetrabutylammonium bromide and 16.0 ml of DMF. Reaction mixture was stirred for 30-60 minutes at 25-30° C. Subsequently, raised the reaction temperature to 30-35° C. and 30 g (0.11 mole) of o-Chlorophenyl-α-bromo methyl acetate was added to the reaction mixture over 1 hour and maintained for 4 hours at 30-35° C. Completion of the reaction was ensured by HPLC analysis. Reaction mixture was then cooled to 30° C. and 100 ml of DM water was added to form a two-phase system and stirred for 0.5-2 hours and two resulting phases were separated. To the aqueous phase 20 ml of toluene was added and the organic layers were combined. To the organic phase, 60 ml of toluene, 10 g of DMF and 12 g of (−)-10-camphorsulfonic acid were added. Reaction mixture was seeded with 0.1 g of CLD-CSA crystals and maintained for 4 hours. Gradually cooled to 15-20° C. and maintained for 4-5 hours, filtered and washed with 2×20 ml of chilled toluene. Then washed with 20 ml of chilled acetone and vacuum dried the compound. Charged wet CLD-CSA salt into a 500 ml reaction vessel equipped with reflux condenser. To this, added 10 volumes of acetone and heated to reflux and maintained for 6 hours. Gradually cooled to 15-20° C., filtered and vacuum dried. Spray washed the wet cake with 50 ml of chilled acetone. Dried the obtained compound at 40-50° C. under reduced pressure to afford 21 g (66.87% yield) of CLD-CSA salt. Optical purity: 99.1%.

### Example 8

### Preparation of (+) Clopidogrel (−) CSA Salt

20 g (0.11 mole) of 4,5,6,7-tetrahydrothieno (3,2-c) pyridine hydrochloride was added to a 500 ml reaction vessel equipped with reflux condenser and over head agitator, to this, 100 ml of ethyl acetate was added at 25-30° C. and charged with 34.8 g (0.25 mole) of potassium carbonate, 16.0 ml of DM water, 1 g of butylated hydroxytoluene. Reaction mixture was stirred for 30-60 minutes at 25-30° C. Subsequently, raised the reaction temperature to 30-35° C. and 30 g (0.11 mole) of o-Chlorophenyl-α-bromo methyl acetate was added to the reaction mixture over 1 hour and maintained for 4 hours at 30-35° C. The completion of reaction was ensured by HPLC analysis. Reaction mixture was then cooled to 30° C. and 200 ml of DM water was added to form a two-phase system and stirred for 0.5-2 hours and two resulting phases were separated. To the aqueous phase, 20 ml of ethyl acetate was added and combined the ethyl acetate layer with the main organic layer. Distilled off ethyl acetate under reduced pressure at 40-60° C. To the organic phase, 160 ml of toluene, 10 g of dimethyl formamide and 12 g of camphorsulfonic acid were added. The reaction mixture was seeded with 0.1 g of CLD-CSA crystals and maintained for 4 hours. Gradually cooled to 15-20° C. and maintained for 4-5 hours then filtered and washed with 2×20 ml of chilled toluene. Then washed with 20 ml of chilled acetone and vacuum dried the compound Charged wet CLD-CSA salt into a 500 ml reaction vessel equipped with reflux condenser. To this, added 10 volumes of acetone and heated to reflux and maintained for 6 hours. Gradually cooled to 15-20° C. and filtered and vacuum dried. Spray washed the wet cake with 50 ml of chilled acetone. Dried the wet CLD-CSA salt at 40-50° C. under vacuum. Yield of CLD-CSA: 21 g (66.87% yield) Optical purity: 99.2%.

### Example 9

### Preparation of (+) Clopidogrel (−) CSA Salt

20 g (0.11 mole) of 4,5,6,7-tetrahydrothieno (3,2-c) pyridine hydrochloride was added to a 1000 ml reaction vessel equipped with reflux condenser and over head agitator, to this, 100 ml of ethyl acetate was added at 25-30° C. and charged with 174 g (0.25 mole) of potassium carbonate, 16.0 ml of DM water, 1 g of TBAB. Reaction mixture was stirred for 30-60 minutes at 25-30° C. Subsequently, raised the reaction temperature to 30-35° C. and 30 g (0.11 mole) of o-Chlorophenyl-α-bromo methyl acetate was added to the reaction mixture over 1 hour and maintained for 4 hour at 30-35° C. The completion of the reaction was ensured by HPLC analysis. Reaction mixture was then cooled to 30° C. and 200 ml of DM water was added to form a two-phase system and stirred for 0.5-2 hours and two resulting phases were separated. To the aqueous phase, 20 ml of ethyl acetate was added and combined the ethyl acetate layer to the main organic layer. Distilled off ethyl acetate under reduced pressure at 40-60° C. To the organic layer, 160 ml of toluene, 10 g of dimethyl formamide and 12 g of camphorsulphonic acid were added. The reaction mixture was seeded with 0.1 g CLD-CSA crystals and maintained for 4 hours. Gradually cooled to 15-20° C. and maintained for 4-5 hours, filtered and washed with 2×20 ml of chilled toluene. Then washed with 20 ml of chilled acetone and vacuum dried the compound. Charged wet CLD-CSA salt into a 500 ml reaction vessel equipped with reflux condenser. To this, added 10 volumes of acetone and heated to reflux and maintained for 6 hours. Gradually cooled to 15-20° C. and filtered and vacuum dried. Spray washed the wet cake with 50 ml of chilled acetone. Dried the wet CLD-CSA salt at 40-50° C. under vacuum. Yield of CLD-CSA: 22 g (70.06% yield) Optical purity: 99.2%.

### Example 10

### Preparation of (+) Clopidogrel (−) CSA Salt

100 g (0.57 mole) of 4,5,6,7-tetrahydrothieno(3,2-c)pyridine hydrochloride was added to a 1000 ml reaction vessel equipped with reflux condenser and over head agitator, to this, 400 ml of toluene was added at 25-30° C. and charged with 174 g (1.25 mole) of potassium carbonate, 80 ml of DM water and 80 ml of DMF. Reaction mixture was stirred for 30-60 minute at 25-30° C. Subsequently, raised the reaction temperature to 30-35° C. and o-Chlorophenyl-α-bromo methyl acetate (0.57 mole) was added to the reaction mixture over 1 hour and maintained for 4 hours at 30-35° C. Ensured the completion of reaction by HPLC analysis. Reaction mixture was then cooled to 30° C. 100 ml of toluene and 500 ml of DM water were added to form a two-phase system and stirred for 30-120 minutes and two resulting phases were separated. Washed the organic layer with 100 ml of DM Water. To the organic phase, 245 ml of toluene was added. 49 ml of DMF was also added and stirred for 30 minutes to obtain a clear solution. To this, charged with 70 g of camphorsulfonic acid and heated to 40° C. 0.1 g of CLD-CSA crystals was then added and maintained for 4 hours. Gradually cooled to 15-20° C. and maintained for 4-5 hours, filtered and washed with 2×100 ml of chilled toluene. Then washed with 100 ml of chilled acetone and vacuum dried the compound. Charged wet CLD-CSA salt into a 2000 ml reaction vessel equipped with reflux condenser. To this, added 10 volumes of acetone and heated to reflux and maintained for 6 hours and gradually cooled to 15-20° C., filtered and vacuum dried. Spray washed the wet cake with 500 ml of chilled acetone. Dried the wet CLD-CSA salt at 40-50° C. under vacuum. Yield of CLD-CSA: 109 g (69.42% yield) Optical purity: 99.3%.

### Example 11

### Preparation of (+) Clopidogrel (−) CSA Salt

20 g (0.11 mole) of 4,5,6,7-tetrahydrothieno-(3,2-c)pyridine hydrochloride was added to a 500 ml reaction vessel equipped with reflux condenser and over head agitator, to this, 80 ml of toluene was added at 25-30° C. and charged with 34.8 g (0.25 mole) of potassium carbonate, 16 ml of DM water, and 20 ml of dimethylsulfoxide. Reaction mixture was stirred for 30-35 minutes at 25-30° C. Subsequently, raised the reaction temperature to 30-35° C. and 30 g (0.11 mole) of o-Chlorophenyl-α-bromo methyl acetate was added to the reaction mixture over 1 hour and maintained for 4 hours at 30-35° C. Completion of the reaction was ensured by HPLC analysis. Reaction mixture was then cooled to 30° C. 20 ml of toluene and 100 ml of DM water were added to form a two-phase system. Stirred the reaction mixture for 0.5-2 hours and two resulting phases were separated. Washed the organic layer with 20 ml of DM water. To the aqueous phase, 20 ml of toluene was added and combined the organic layers. To the organic layer, 60 ml of toluene, 10 g of DMF and 12 g of camphorsulphonic acid were added. The reaction mixture was seeded with 0.1 g crystals of CLD-CSA and maintained for 4 hours. Gradually cooled the reaction mixture to 15-20° C. and maintained for 4-5 hours, filtered and washed with 2×20 ml of chilled toluene. Then washed with 20 ml of chilled acetone and vacuum dried the compound. Charged wet CLD-CSA salt into a 500 ml reaction vessel equipped with reflux condenser. To this, added 10 volumes of acetone and heated to reflux and maintained for 6 hours. Gradually cooled to 15-20° C. and filtered and vacuum dried. Spray washed the wet cake with 50 ml of chilled acetone. Dried the obtained compound at 40-50° C. under reduced pressure to afford 20 g (63.69% yield) of CLD-CSA salt. Optical purity: 99.3%.

### Example 12

### Preparation of (+) Clopidogrel (−) CSA Salt

20 g (0.11 mole) of 4,5,6,7-tetrahydrothieno-(3,2-c)pyridine hydrochloride was added to a 500 ml reaction vessel equipped with reflux condenser and over head agitator, to this, 80 ml of toluene was added at 25-30° C. and charged with 34.8 g (0.25 mole) of potassium carbonate, 16 ml of DM water, and 20 ml of dimethylacetamide. Reaction mixture was stirred for 30-35 minutes at 25-30° C. Subsequently, raised the reaction temperature to 30-35° C. and 30 g (0.11 mole) of o-Chlorophenyl-α-bromo methyl acetate was added to the reaction mixture over 1 hour and maintained for 4 hours at 30-35° C. Completion of the reaction was ensured by HPLC analysis. Reaction mixture was then cooled to 30° C. and added 20 ml of toluene and 100 ml of DM water to form a two-phase system. Stirred the reaction mixture for 0.5-2 hours and two resulting phases were separated. Washed the organic layer with 20 ml of DM water. To the aqueous phase, 20 ml of toluene was added and the organic layers were combined. To the organic layer, 60 ml of toluene, 10 g of DMF and 12 g of camphorsulfonic acid were added. Also added 0.1 g crystals of CLD-CSA and maintained for 4 hours. Gradually cooled the reaction mixture to 15-20° C. and maintained for 4-5 hours, filtered and washed with 2×20 ml of chilled toluene. Then washed with 20 ml of chilled acetone and vacuum dried the compound. Charged wet CLD-CSA salt into a 500 ml reaction vessel equipped with reflux condenser. To this, added 10 volumes of acetone and heated to reflux and maintained for 6 hours. Gradually cooled to 15-20° C. and filtered and vacuum dried. Spray washed the wet cake with 50 ml of chilled acetone. Dried the obtained compound at 40-50° C. under reduced pressure to afford 21 g (66.87% yield) of CLD-CSA salt. Optical purity: 99.2%.

### Example 13

### (S)-Clopidogrel Camphor Sulfonate Recovery from Mother Liquor

815 ml of mother liquor from purification of CLD-CSA in acetone of example 1 was distilled atmospherically to obtain crude solid. 960 ml of mother liquor from CLD resolution stage in toluene of example 1 was added to the above crude solid. 480 ml of water was added to form a two-phase system and adjusted the pH to 8-8.5 with sodium bicarbonate powder at 30-35° C. Two resulting phases were separated and the organic phase was washed with 480 ml of water. The organic phase was dried by azeotropic distillation under reduced pressure until KF (Karl Fisher)<0.1%. Cooled the reaction mixture to 15-20° C. and 6 g of potassium tert-butoxide was added. Aged the reaction mixture for 45-60 minutes and 500 ml of water was added and stirred for 30 minutes at 20-25° C. The organic layers were separated and washed with 150 ml of 10% sodium chloride solution. Ensured the moisture of organic layer was less than 0.1%. Subsequently, 29.5 g of DMF and 42.5 g of camphorsulfonic acid were added. Seeded with 0.2 g of CLD-CSA crystals and maintained for 4 hours. Gradually cooled the reaction mixture to 15-20° C. and maintained for 4-5 hours, filtered and washed with 2×41 ml of chilled toluene and vacuum dried the compound. Charged wet CLD-CSA salt into a 1000 ml reaction vessel equipped with reflux condenser. To this, added 10 volumes of acetone and heated to reflux and maintained for 6 hours. Gradually cooled to 15-20° C. and filtered and vacuum dried. Spray washed the wet cake with 2×44 ml of chilled acetone. Dried the obtained compound at 40-50° C. under reduced pressure to afford 65 g (92.8% yield) of CLD-CSA salt. Chiral purity: 100%.

### Example 14

### Preparation of Clopidogrel Bisulfate Form I from the Camphor Sulfonate Salt

CLD-CSA salt was added to ethyl acetate and water. Then sodium hydroxide was charged to the batch followed by sodium bicarbonate. The organic phase was separated from the aqueous phase and washed with water, and then decolorized with charcoal. Once the charcoal was filtered, the batch was concentrated. The obtained residue was then dissolved in acetone, and then sulfuric acid and clopidogrel polymorph seed were added. The crystals were aged while stirring, subsequently, filtered and washed with acetone. The crystals were then dried under vacuum at a temperature of less than 25° C.

What is claimed is:

1. A process for preparing (−)-10-camphorsulphonic acid salt of methyl (+)-(S)-α-(2-chlorophenyl)-6,7-dihydrothieno[3,2-c]pyridine-5(4H)-acetate comprising: (a) reacting 4,5,6,7-tetrahydrothieno (3,2-c) pyridine hydrochloride with o-chlorophenyl-α-bromo methyl acetate in the presence of an acid acceptor to produce (±)-methyl α-(2-chlorophenyl)-6,7-dihydrothieno[3,2-c]pyridine-5(4H)-acetate; and (b) reacting in-situ (±)-methyl α-(2-chlorophenyl)-6,7-dihydrothieno[3,2-c]pyridine-5(4H)-acetate with (−)-10-camphorsulphonic acid to provide (−)-10-camphorsulphonic acid salt of methyl (+)-(S)-α-(2-chlorophenyl)-6,7-dihydrothieno[3,2-c]pyridine-5(4H)-acetate.

2. The process of claim 1, wherein the acid acceptor is an inorganic base selected the group consisting of alkali metal carbonate and bicarbonate.

3. The process of claim 2, wherein the acid acceptor is sodium carbonate, potassium carbonate, sodium bicarbonate or potassium bicarbonate.

4. The process of claim 1, wherein the acid acceptor is employed in amounts ranging from about 1 mole to about 4 moles per one mole of 4,5,6,7-tetrahydrothieno-(3,2-c)pyridine hydrochloride.

5. The process of claim 1, wherein the acid acceptor is employed in amounts ranging from about 1.5 moles to about 1.7 moles per one mole of 4,5,6,7-tetrahydrothieno-(3,2-c)pyridine hydrochloride.

6. The process of claim 1, wherein the o-chlorophenyl-α-bromo methyl acetate is present in amounts ranging from about 0.8 moles to about 1.5 moles per one mole of 4,5,6,7-tetrahydrothieno (3,2-c) pyridine hydrochloride.

7. The process of claim 1, wherein the reaction between 4,5,6,7-tetrahydrothieno (3,2-c) pyridine hydrochloride and o-chlorophenyl-α-bromo methyl acetate is carried out at a temperature of about 25° C. to about 100° C.

8. The process of claim 1, wherein the reaction between 4,5,6,7-tetrahydrothieno (3,2-c) pyridine hydrochloride and o-chlorophenyl-α-bromo methyl acetate is carried out at a temperature of about 25° C. to about 60° C.

9. The process of claim 1, wherein the reaction between 4,5,6,7-tetrahydrothieno (3,2-c) pyridine hydrochloride and o-chlorophenyl-α-bromo methyl acetate is carried out in a biphasic solvent system.

10. The process of claim 9, wherein the biphasic solvent comprises water and water-immiscible organic solvent.

11. The process of claim 10, wherein the water-immiscible organic solvent is selected from the group consisting of C₆ to C₁₂ aromatic hydrocarbons, halogenated hydrocarbons, C₃ to C₈ ketone, C₃ to C₁₀ alkyl ester, and mixtures thereof.

12. The process of claim 10, wherein the water immiscible-organic solvent in the biphasic solvent is present in amounts ranging from about 2 ml to about 10 ml per gram of 4,5,6,7-tetrahydrothieno (3,2-c) pyridine hydrochloride.

13. The process of claim 9, wherein the water is present in amounts ranging from about 0.5 volumes to about 5 ml per gram of 4,5,6,7-tetrahydrothieno (3,2-c) pyridine hydrochloride.

14. The process of claim 9, wherein the biphasic solvent further comprises a co-solvent selected from the group consisting of dimethyl formamide, dimethyl sulfoxide, toluene, heptane and dimethylacetamide.

15. The process of claim 14, wherein the co-solvent is present in amounts ranging from about 0.2 to about 1 ml per gram of 4,5,6,7-tetrahydrothieno (3,2-c) pyridine hydrochloride.

16. The process of claim 1, wherein the reaction between 4,5,6,7-tetrahydrothieno (3,2-c) pyridine hydrochloride and o-chlorophenyl-α-bromo methyl acetate is carried out under phase transfer conditions.

17. The process of claim 16, wherein the process further includes a phase transfer catalyst.

18. The process of claim 17, wherein the phase transfer catalyst is selected from the group consisting of quaternary ammonium salts, phosphonium salts, crown ethers, and pyridium salt.

19. The process of claim 17, wherein the phase transfer catalyst is a quaternary ammonium salt.

20. The process of claim 17, wherein the phase transfer catalyst is employed in amounts ranging from about 0.01 mole to about 0.1 mole per one mole of 4,5,6,7-tetrahydrothieno-(3,2-c)pyridine hydrochloride.

21. A process for preparing a pharmaceutically acceptable salt of (S)-clopiogrel, further comprising converting (−)-10-camphorsulphonic acid salt of methyl (+)-(S)-α-(2-chlorophenyl)-6,7-dihydrothieno[3,2-c]pyridine-5(4H)-acetate of claim 1 to a pharmaceutically acceptable salt of (S)-clopiogrel.

22. The process of claim 21, wherein the pharmaceutically acceptable salt is bisulfate.

23. A process for preparing (S)-clopidogrel (−)-10-camphorsulphonic acid salt comprising: (a) combining (R) clopidogrel or a mixture of (R) and (S) clopidogrel with a base to obtain a racemic mixture of (R) and (S) clopidogrel further enriched with (S) clopidogrel; and (b) reacting the racemic mixture of (R) and (S) clopidogrel further enriched with (S) clopidogrel with levorotatory camphorsulphonic acid, to provide (S)-clopidogrel (−)-10-camphorsulphonic acid salt, wherein steps (a) and (b) are carried out without an intermediate step of reacting the racemic mixture of (R) and (S) clopidogrel with sulfuric acid.

24. The process of claim 23, further comprising recrystallizing (S)-clopidogrel (−)-10-camphorsulphonic acid salt in a suitable organic solvent.

25. The process of claim 23, wherein the base is an organic amine, an alkali metal alkoxide, an alkali metal hydroxide, an alkaline earth metal hydroxide, an alkali metal hydride, an alkaline earth metal hydride, an alkali or alkaline earth metal carbonate or hydrogencarbonate salt.

26. The process of claim 23, wherein the base is sodium hydroxide, potassium hydroxide, sodium C₁ to C₄ alkoxide or potassium C₁ to C₄ alkoxide.

27. The process of claim 23, wherein the base is sodium t-butoxide or potassium t-butoxide.

28. The process of claim 23, wherein the base is present in amounts ranging from about 0.01 to about 0.5 mole per one mole of (R) clopidogrel or a mixture of (R) and (S) clopidogrel.

29. The process of claim 23, wherein prior to step (a), the process comprises combining a mother liquor of (R) clopidogrel (−)-10-camphorsulphonic acid salt or a mixture of (R) and (S) clopidogrel (−)-10-camphorsulphonic acid salt with a base in an organic solvent to obtain (R) clopidogrel or a mixture of (R) and (S) clopidogrel.

30. The process of claim 29, wherein the base is an organic amine, an alkali metal alkoxide, an alkali metal hydroxide, an alkaline earth metal hydroxide, an alkali metal hydride, an alkaline earth metal hydride, an alkali or alkaline earth metal carbonate or hydrogencarbonate salt.

31. The process of claim 29, wherein the base is sodium hydrogen carbonate.

32. The process of claim 29, wherein the base is employed in amounts ranging from about 0.1 to 1.0 mole per 1 liter of mother liquor.

33. The process of claim 29, wherein the organic solvent is selected from the group consisting of C₆ to C₁₂ aromatic hydrocarbons, halogenated hydrocarbons, C₃ to C₈ ketone, C₃ to C₁₀ alkyl ester, and mixtures thereof.

34. The process of claim 23, wherein the process further comprises converting (S)-clopidogrel (−)-10-camphorsulphonic acid salt to a pharmaceutically acceptable salt of (S)-clopiogrel.

35. The process of claim 34, wherein the pharmaceutically acceptable salt is bisulfate.

36. A process for preparing clopidogrel camphosulfonate comprising combining 4,5,6,7-tetrahydrothieno-(3,2-c)pyridine hydrochloride, toluene, dimethyl formamide, o-chlorophenyl-α-bromo methyl acetate to obtain a reaction mixture containing (±) clopidogrel; and converting (±) clopidogrel to clopidogrel camphosulfonate without the recovery of (±) clopidogrel.

37. The process of claim 36, wherein the process further comprises adding tetrabutylammonium hydrogen sulphate and/or a base to the combination of 4,5,6,7-tetrahydrothieno-(3,2-c)pyridine hydrochloride, toluene, dimethyl formamide, and o-chlorophenyl-α-bromo methyl acetate.

38. A process for preparing a pharmaceutically acceptable salt of (S)-clopiogrel, further comprising converting the (−)-10-camphorsulphonic acid salt of methyl (+)-(S)-α-(2-chlorophenyl)-6,7-dihydrothieno[3,2-c]pyridine-5(4H)-acetate of claim 36 to a pharmaceutically acceptable salt of (S)-clopiogrel.

39. A process for preparing (S)-clopidogrel bisulfate comprising:
(a) reacting 4,5,6,7-tetrahydrothieno (3,2-c) pyridine hydrochloride with o-chlorophenyl-α-bromo methyl acetate in the presence of an acid acceptor to produce (±)-methyl α-(2-chlorophenyl)-6,7-dihydrothieno[3,2-c]pyridine-5(4H)-acetate;
(b) reacting in-situ (±)-methyl α-(2-chlorophenyl)-6,7-dihydrothieno[3,2-c]pyridine-5(4H)-acetate with (−)-10-camphorsulphonic acid to provide (−)-10-camphorsulphonic acid salt of methyl (+)-(S)-α-(2-chlorophenyl)-6,7-dihydrothieno[3,2-c]pyridine-5(4H)-acetate, wherein the camphorsulfonate precipitates out of reaction mixture leaving (R)-clopidogrel in the reaction mixture (mother liquor), optionally in a mixture with lesser amounts of (S)-clopidogrel;
(c) combining the (R) clopidogrel or the mixture of (R) and (S) clopidogrel remaining in the reaction mixture (mother liquor) with a base to obtain a racemic mixture of (R) and (S) clopidogrel further enriched with (S) clopidogrel;
(d) reacting the racemic mixture of (R) and (S) clopidogrel further enriched with (S) clopidogrel with levorotatory camphorsulphonic acid, to provide (S)-clopidogrel (−)-10-camphorsulphonic acid salt, wherein steps (c) and (d) are carried out without an intermediate step of reacting the racemic mixture of (R) and (S) clopidogrel with sulfuric acid; and
(e) converting (S)-clopidogrel (−)-10-camphorsulphonic acid salt to (S)-clopidogrel bisulfate.
